(19) United States
(12) Patent Application Publication
(10) Pub. No.: US 2014/0000613 A1
(43) Pub. Date: Jan. 2, 2014

(54) Apparatus, System and Method of Remotely Actuating a Manual Ventilation Bag

(71) Applicant: Andrew Hines, Boonton, NJ (US)

(72) Inventor: Andrew Hines, Boonton, NJ (US)

(21) Appl. No.: 13/922,918

(22) Filed: Jun. 20, 2013

Related U.S. Application Data
(60) Provisional application No. 61/661,977, filed on Jun. 20, 2012.

(51) Int. Cl. A61M 16/00

(52) U.S. Cl. CPC A61M 16/0078
USPC 128/205.16

(57) ABSTRACT

An apparatus, system and method of actuating a manual ventilator system bag to cause patient ventilation. The apparatus, system and method include a pressure exerter proximate to the manual ventilator system bag; and an actuator suitable for remote actuation of said pressure exerter. In embodiments, actuation of the actuator remotely causes the pressure exerter to make contact with and exert pressure against the manual ventilator system bag to thereby execute ventilation of the patient associated with an inlet hose/tube.

### CROSS REFERENCE TO RELATED APPLICATIONS

This application claims priority to U.S. Provisional Patent Application Ser. No. 61/661,977, filed Jun. 20, 2012, entitled Apparatus, System and Method of Remotely Actuating a Manual Ventilation Bag, the entirety of which is expressly incorporated herein by reference in its entirety.

### FIELD OF THE INVENTION

The field of the present invention relates to an apparatus, system and method for aiding in patient ventilation, and, more particularly to an apparatus, system and method of remotely actuating a manual ventilation bag.

### BACKGROUND OF THE INVENTION

Historically, ventilator systems, also referred to hereinthroughout as anesthetic breathing systems or circuits, are broadly classified as “closed” or “open,” which classifications are differentiated based on the whether and to what extent “rebreathing” is employed. In a “circular” rebreathing system, rebreathing is used, which leads to a circular, closed or semi-closed flow of the breathing circuit. That is, the flow of gases is provided into and out of the patient in a closed or semi-closed loop, and is moderated by valves so as to prevent backflow of gases in an improper direction. For example, a fully circular, or closed, system may typically use a “reservoir bag,” and the gases may flow from the reservoir bag to an inhalation valve to an inhalation hose to the patient, and then from the patient to an exhalation hose to an exhalation valve to a CO2 canister and back to the inhalation valve. In such a system, the CO2 is treated, such as by washout with adequate fresh gas flow (FGF) or by soda lime absorption, to allow for use of the exhaled air, in part, in the rebreathing process. An exemplary rebreathing circuit is shown in the illustration of FIG. 1.

In a non-rebreathing system, certain of which are often classified using the “Mapleson” classification, a “breathing bag” is similarly employed in order to aid the patient in the breathing process. However, a non-rebreathing system is typically open or semi-open, meaning that the recycling of the exhalation of the patient is either not performed, or is performed to a far lesser extent than in a rebreathing system. The Mapleson circuits are illustratively provided in FIG. 2 by Mapleson classification, and, of note, only a Mapleson type E does not employ a breathing bag.

Regardless of type, any breathing circuit delivers oxygen (O2) and/or anesthetic gases to, and must eliminate carbon dioxide (CO2) expelled from, the patient. This is most frequently accomplished through the inclusion of the aforementioned reservoir, or breathing, bag in the ventilation circuit. The bag is typically made of antistatic rubber or plastic, and is typically of a volume between 1 L and 30 L. The bag accommodates fresh gas flow during expiration, such as by acting as a reservoir available for use of the following inspiration in a rebreathing system. Further, the bag may be used to assist in or control the patient's ventilation. For example, in certain systems the bag may be manually actuated, and may provide a system delimiter for pressure in the breathing circuit.

In addition to the foregoing bags, a ventilator may include, within the circuit, a manual breathing bag, which may allow for a caregiver to manually take over the otherwise automated function of the ventilator. This manual bag may, in some instances, serve as a substitute for an automated ventilator bag, or, in other cases, may be one in the same with the afore-discussed reservoir bag or breathing bag. In the discussion herein, reference to a “bag” or “breathing bag” indicates reference to all applicable ones, in a given example, from among the reservoir, breathing, and manual bags.

The breathing bag, in addition to the other components typical of a breathing circuit, is typically connected to breathing circuit components and to/from the patient through a series of hoses. However, the use of hoses is counterproductive in the breathing circuit, in part because the hoses add resistance to the breathing circuit. This resistance is related to the length and radius of the hoses, and additionally to the viscosity of the gas flowing through the hose, by the equation:

$$Resistance = \frac{Length \times Viscosity}{{Radius}^{4}}$$

Therefore, it is undesirable to add additional or unnecessary hoses, or additional bends or narrowing of the radius of the hose, even to increase the convenience of the health care professionals treating the anesthetized patient. Such additions or bends to hosing can increase system resistance, modifying the desired system pressure and making it more difficult for the patient to breathe and/or to breathe in a proper mixture. Consequently, it is frequent that a doctor or anesthesiologist must inconveniently access the breathing bag when necessary, such as by reaching outward from a necessary location and thereby removing at least one hand from the task at hand, or worse yet such as by having to move from the necessary location to actuate, by hand, the bag as needed before returning to the necessary location.

Thus, the need exists for an apparatus, system and method of providing patient ventilation that increases the convenience of the caregiver in ventilating the patient using a breathing bag, but that does not provide increased resistance or allow for pressure variations in the breathing system.

### BRIEF DESCRIPTION OF THE FIGURES

Embodiments of the present invention will be understood with reference to the detailed description in conjunction with the accompanying figures, in which like numerals indicate like aspects, and wherein:

FIG. 1 illustrates a ventilation system in accordance with the known art;

FIG. 2 illustrates a plurality of non-rebreathing ventilation systems;

FIG. 3 illustrates a perspective view of an apparatus in accordance with the present invention;

FIG. 4 illustrates side view of an apparatus in accordance with the present invention;

FIG. 5 illustrates a side view of an apparatus in accordance with the present invention;

FIGS. 6A-B illustrates a perspective view of an embodiment of an apparatus in accordance with the present invention; and

FIG. 7 illustrates a flow of a method according to the present invention.

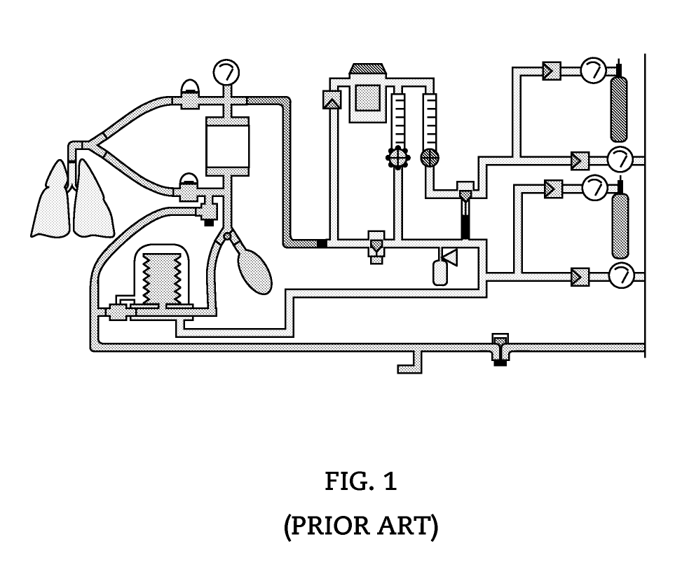

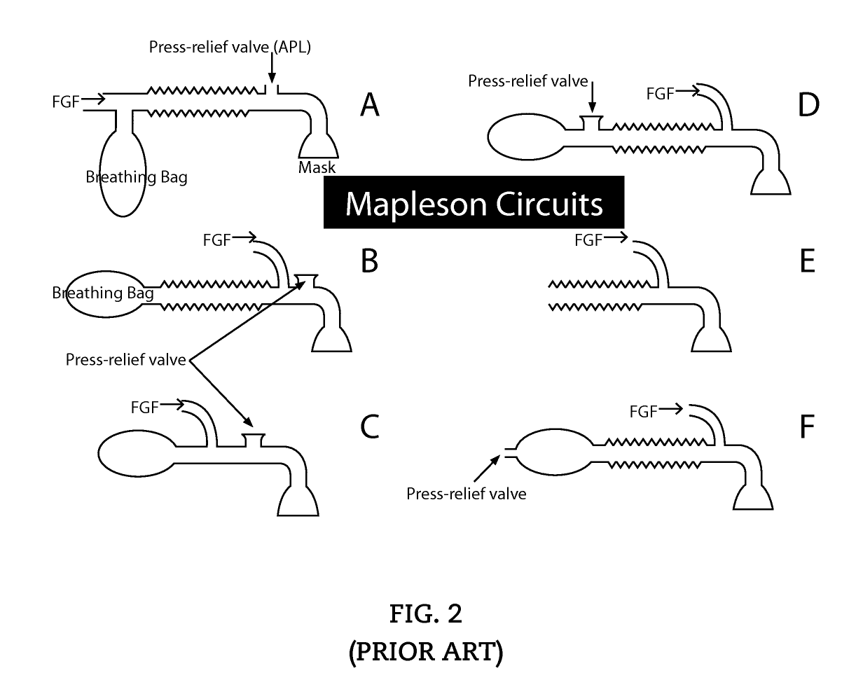

### SUMMARY OF THE INVENTION

The present invention is and includes at least an apparatus, system and method of actuating a manual ventilator system bag to cause patient ventilation. The apparatus, system and method include a bag mount suitable for maintaining a position of the manual ventilator system bag and at least an inlet house associated with the manual ventilator system bag; a pressure exerter proximate to the manual ventilator system bag; and an actuator suitable for remote actuation of said pressure exerter. In embodiments, actuation of the actuator remotely causes the pressure exerter to make contact with and exert pressure against the manual ventilator system bag to thereby execute ventilation of the patient associated with the inlet hose.

In certain exemplary embodiments, the pressure exerter may be pressure paddles aside the bag, or an air-filled ring around the bag. Further, the actuator may be, for example, a pedal, a button, or a handle. The actuator may send the actuation indication to the pressure exerter via, for example, a cable within a sheath traversing the distance between the bag mount and the actuator. Use of the actuator eliminates the need for a doctor, nurse or anesthetist to remove a hand, or be moved in the entirety, from the task at hand. In fact, even if the pedal is not reachable by someone other than the one with the pedal, the open design permits anyone to reach onto the bag and manually squeeze as necessary in the same manner as always performed prior to the invention.

Thus, the present invention provides at least an apparatus, system and method of providing patient ventilation that increases the convenience of the caregiver in ventilating the patient using a breathing bag, but that does not provide increased resistance or allow for pressure variations in the breathing system. Moreover, patient safety is greatly increased by providing a means to ventilate, without removing hands from the mask and/or the patients body.

### DETAILED DESCRIPTION OF THE EMBODIMENTS

It is to be understood that the figures and descriptions of the present invention have been simplified to illustrate elements that are relevant for a clear understanding of the present invention, while eliminating, for the purpose of clarity and brevity, many other elements found in typical ventilation apparatuses, systems and methods. Those of ordinary skill in the art may thus recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to the disclosed elements and methods known to those skilled in the art.

There are a variety of types of ventilation systems that require extremely precise control over, for example, patient flow volume, ventilation pressure, and the like. In the event that these system criteria break down, a caregiver may be alerted that the required criteria for proper ventilation have been violated, and manual ventilation may be necessary.

For example, ventilators typically offer volume controlled ventilation (VCV). In VCV, a set volume of gas is preferably delivered at a constant flow rate. However, the peak inspiratory pressure may vary according to the patient's compliance and airway resistance. In VCV, the volume may preferably be at 5-25 mL/kg, and the breathing rate may be at 5-20 breaths per minute (bpm).

Synchronized intermittent mandatory volume controlled ventilation (SIMV-VCV detects spontaneous breaths (if any) of the patient and delivers ventilator-generated breaths in synchronization with the patient's inspiratory efforts. If too many (or too few) synchronized breaths are delivered, the ventilation trigger or sensitivity, or manual efforts to assist with ventilation, may be modified.

In pressure control ventilation (PCV), inspiratory pressure is preferably controlled, allowing the inspired volume to vary, such as in compliance with changes in airway resistance. Flow may be high at first, to produce a set pressure early in inspiration, and less later to maintain the set pressure through the inspiratory time.

These and other ventilator types may include alarms when, for example, high pressure or volume, low pressure or volume, high or low respiratory rate, reverse flow through a unidirectional valve, or apnea or breathing disconnect occurs, and such alarms may be audio, visual, automated or manual. Upon occurrence of an automated alarm, or, for example, upon occurrence of the failure of the breathing bag to properly inflate, manual assistance may be provided to the patient, such as by squeezing the breathing bag at a predetermined pressure and at a predetermined rate. When this manual actuation is necessary, it preferably is performed without removal of the caregiver, or of the caregiver's hand(s), from a necessary position, and without inconveniencing the caregiver in the necessary position, and further without need of extended hosing to and from the manual breathing bag, such as to a valve mounted in some other position or location.

The present invention is and includes an apparatus, such as an actuator, for manually compressing a breathing bag in a ventilation system. In certain preferred embodiments, the actuation of the bag is responsive to actuation of a foot pedal, although in alternative embodiments the actuation of the bag may be responsive to other mechanical devices, such as responsive to the pressing of a button, pulling or squeezing of a handle, or the like. It is necessary in the present invention that the mechanical manual actuation of the bag be non-intrusive as to the integrity of the ventilation system, and that the mechanical actuation be adjustable to account for system parameters, such as allowable volume, pressure, gas mix or the like. Such adjustments to the available input, output, or throughput of the bag responsive to the mechanical actuation may be made manually, such as by adjusting screws, sliders, switches, stops, or the like, that may delimit the actuation of the bag by the mechanical actuator; or may be made automatically, such as by adjustments to the spring tensions, wire tension, mechanical stops, or the like, made responsively to input to a computing system, such as a personal computer or a programmable logic controller, by way of non-limiting example.

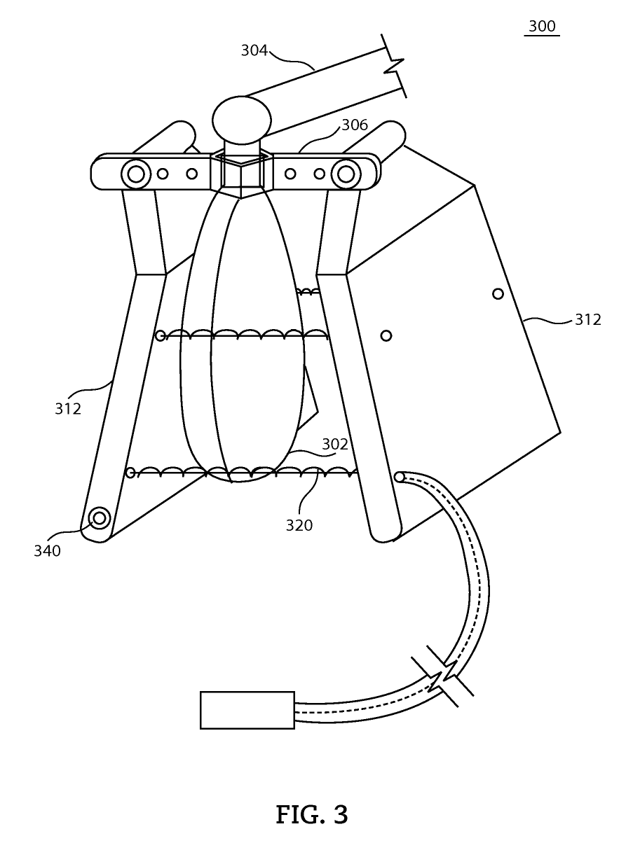

FIG. 3 is a schematic diagram illustrating an exemplary embodiment of a foot pedal actuated manual breathing bag apparatus 300 in accordance with the present invention. In the illustrated embodiment, a tube 304 is connected atop the breathing bag 302, as discussed hereinthroughout, which tube 304, may be, for example, for inhalation and/or exhalation gases to be carried to/from the patient. In an alternative embodiment of the present invention, the bag 302 may be attached to and/or suspended from a rigid tube, the tube being part of the delivery system and may be utilized to provide temporary “supplemental” or “additional” pressure into the system. Further illustrated is one or more mounting means 306, such as the illustrated metallic or plastic brace that allow for the bag 302 to be sturdily mounted proximate to the mechanical actuating mechanism, such as in a suspended manner, to allow for avoidance of dislodging of the bag 302 or the incoming tube 304 or hoses.

In the embodiment of FIG. 3, further illustrated on each side of the bag 302 are pressure pads or paddles 312 that, upon actuation, apply pressure to the bag 302, wherein such pressure is preferably delimited as referenced hereinabove. Further, as illustrated here and in other exemplary embodiments, the pressure paddles may be rotationally or otherwise movably mounted to either the mount for the bag or to a secondary mount that maintains the position of the pressure paddles in the desired proximity to the bag.

Further illustrated in FIG. 3 is one or more springs 320 that, upon actuation, may allow for exertion of pressure by the pressure paddles to the breathing bag, and that further may have spring constants or provide a desired inverse pressure such that no more than the desired pressure is applied to the breathing bag. As used herein, the inverse pressure may imply that the actuation compresses, rather than expands, the spring, such that once the actuation is relieved the spring decompresses to return the paddles to a position not exerting, or exerting less, pressure on the bag. Although any number of springs may be included in exemplary embodiments such as that of FIG. 3, it may be preferable that at least two springs, and, as in the illustrated embodiment, three or four springs, be included in the mechanical actuator. It is also intended that each individual user may be provided with the ability to “dial in” or adjust the back pressure from, for example, the springs so as to suit the user's preferences or the patient's needs. The operator may also be offered an assortment of different springs. Such springs might be uniformly color coded and relate to the strength of each spring. In this manner, some users might always use, for example, the “red” springs.

The actuation of the springs to apply the pressure paddles to exert pressure upon the bag is responsive to the illustrated actuator (such as a foot pedal) 330. As mentioned above, the term foot pedal is similarly used to reference other like mechanical means capable of indicating desired actuation of the bag, such as a handle or a button. In the illustrated embodiment, the actuation of the foot pedal or like mechanical means may draw an actuator to actuate, i.e., compress as illustrated, springs to exert the pressure paddles upon the sides of the bag. As will be understood by those skilled in the art, the foot pedal actuation may cause a cable 380 sheathed within a line running from the foot pedal to the pressure paddles to be pulled to affect the actuation as shown. Additionally and alternatively, actuation may cause the passing of air, electricity, or the like through a valve or switch to indicate actuation of the pedal and to thereby force actuation of the pressure paddles on the sides of the bag, or the like.

As will be understood by those skilled in the art, the foot pedal may be formed of any reasonably sturdy substance, such as plastic or metal, and may be connected to or via a sheathed cable or other sheath having therein the aforementioned air lines or electricity lines. In certain embodiments, the sheathed cable may be formed of plastic, anti-static rubber, or the like, by way of non-limiting example. Further, the springs discussed with reference to FIG. 3 may be of any make-up, although certain metals may be preferred for the springs. The pressure paddles may be additionally formed of any preferred substance, such as a light-weight, high strength plastic, a light-weight metal, or the like that will allow for continued and repeated exertion of pressure to the sides of the bag without bending, buckling, or like decay of the pressure paddles.

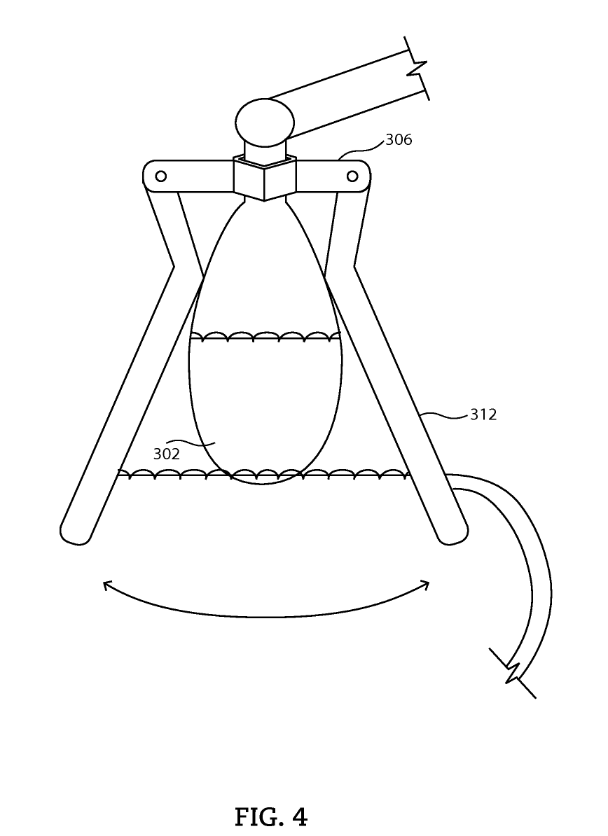

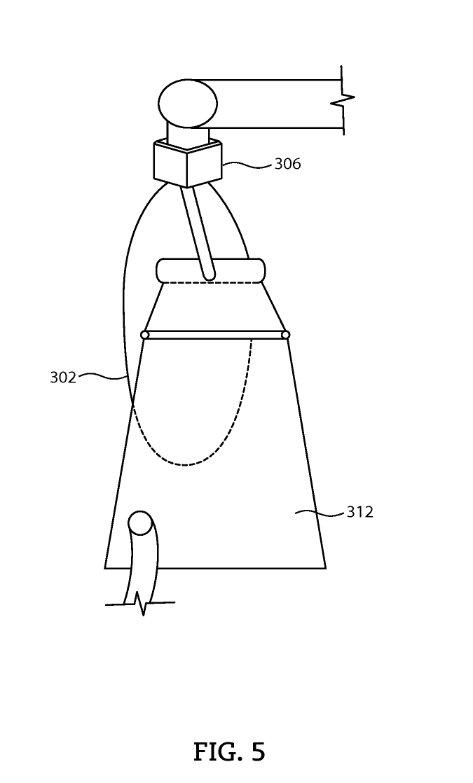

In additional embodiments, the mechanical bag actuator of FIG. 3 may include an emergency release mechanism 340 and may additionally be formed such that the pressure paddles provide a sufficient volume between the pressure paddles to allow for insertion of a caregiver's hand, by way of example, to manually actuate the bag in the event of failure of the mechanical actuator. FIG. 4 shows a side-view of the mechanical actuator of FIG. 3, and FIG. 5 shows an additional side-view of the mechanical actuator of FIG. 3. It is also intended that the caregiver may be enabled, at all times, to simply disengage the entire cable mechanism, thereby returning the system to its original operation.

Those skilled in the art will appreciate that the pressure paddles may take other forms that may be actuated by spring or cable. For example, the pressure paddles may have a curvature imparted thereto, either concave or convex, for ultimate physical association of part or all of the curvature with the sides of the breathing bag; and/or the paddles may include a cylinder or partial cylinder that may be provided about the bag, which cylinder may expand or contract in conjunction with the actuation of the spring or cable running between the desired pressure points in association with the breathing bag and the actuator, such as the pedal. Further, for example, a trunion 340 may be inset to the pressure paddles, such as to allow for stoppage of actuation and/or removal of springs, and the like.

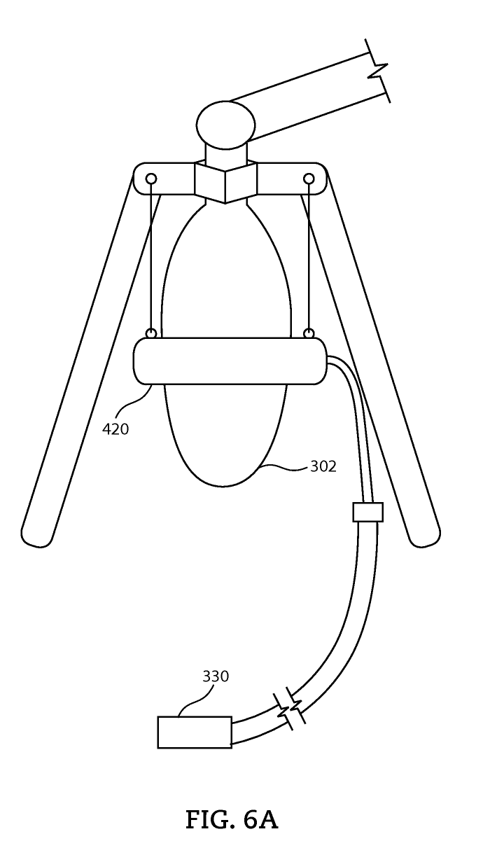

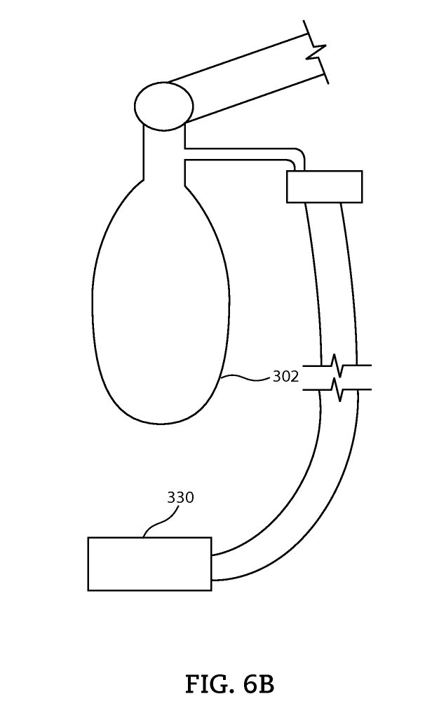

FIG. 6A illustrates an additional exemplary embodiment of the mechanical exertion of pressure on a breathing bag. As is the case with respect to the exemplary embodiments of FIGS. 3-5, FIG. 6A may allow for mechanical exertion of pressure on the breathing bag responsive to actuation of a foot pedal, handle, button, or the like. In the exemplary embodiment of FIG. 6A, actuation of the illustrated foot pedal exerts air pressure on a valve that, when actuated, causes a contraction of a pressure exerter 420 to exert pressure on the breathing bag 302. For example, the illustrated pressure exerter may be an air-filled tube about the circumference of the breathing bag that, when filled with air responsive to the opening of the valve by the foot pedal, causes the contraction of the breathing bag. Likewise, the valve in the embodiment of FIG. 6A may actuate pressure paddles of the shape discussed hereinabove with respect to FIG. 3, or may actuate a pressure exerter having any shape known those skilled in the pertinent art.

Further, those skilled in the art will appreciate, particularly in view of the exemplary embodiments of FIGS. 3-6A, that other mechanically actuated means, such as may be mechanically actuated by a foot pedal, may be used to compress the breathing bag. For example, the foot pedal may include therein one or more switches that, upon actuation of the foot pedal, sense the actuation of the foot pedal and send one or more electrical signals to a means of exerting pressure on the breathing bag. For example, electrically responsive pinchers or the like may be responsive to the electrical signal and may exert pressure on the breathing bag. Such pincers, or the like, may or may not be responsive to proportional controllers or similar means.

Thereby, the present invention provides a means for mechanically actuating a breathing bag relatively remote from the breathing bag. Consequently, a caregiver, not immediately proximate to the breathing bag need not reach out for the breathing bag in order to compress the breathing bag and need not leave a necessary care-giving location to get to and compress the breathing bag. Rather, the controller of the actuation of the breathing bag, such as the pedal, as discussed above, may be moved to any desired location at any time, during, for example, a surgical effort. In such a circumstance, as a surgeon or nurse moves around an operating table, the pedal may be freely moved about the operating table as well in conjunction with the surgeon, nurse or anesthetist, such that the caregiver need not stop the activity being undertaken in order to compress the breathing bag. Further, this actuation of the bag may occur in a manner, due to the nature of the present invention that precludes interference with, knocking into, or otherwise dislodging equipment in the care-giving environment, such as within a surgical room.

Of course, those skilled in the art will appreciate, in light of the illustrations of FIGS. 3-6A, that the present invention would be similarly operable in embodiments not including a bag and/or a means of actuating a bag, but rather including a gas inlet and outlet to be actuated via a valve. For example, in such embodiments and as illustrated in the exemplary embodiment of FIG. 6B, the actuator may be a pedal that simply controls an air flow, or like signaling manner, to a valve. As such, upon actuation of the pedal, an air flow or the like may cause a valve to open to a gas inlet or outlet, thereby enabling ventilation to occur. Thus, an input, such as to pressurize the air flow discussed herein, may be provided to the valve such that, when opened, the valve provides for flow, and thus pressurization, into the system.

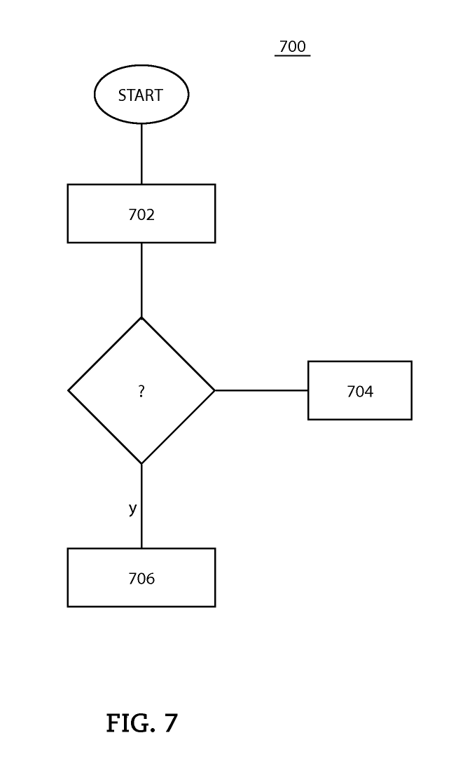

FIG. 7 is a flow diagram illustrating a method in accordance with the present invention. As illustrated, a method 700 according to the present invention may include the step 702 of providing at least one actuatable mechanism in physical association with a breathing bag, and the step 704 of providing, in remote connection, either mechanically (including via air) or electrically, with the actuatable mechanism of an actuator, such as a pedal. Steps 702 and 704 are performed such that, upon actuation of the actuator, the exertion of pressure by the actuatable mechanism on the breathing bag occurs at step 706.

It will be understood that the embodiments of the present invention that are illustrated and described are merely exemplary, and that a person skilled in the art may thus make many variations and modifications thereto. Therefore, all such embodiments, variations and modifications are intended to be included within the scope of the present invention as defined by the description and any claims set forth herein.

What is claimed is:

1. An actuator system for a manual ventilator system bag, comprising:
a mount suitable for maintaining a position of the manual ventilator system bag and at least an inlet hose, tube or cable associated with the manual ventilator system bag;
a pressure exerter proximate to the manual ventilator system bag; and
an actuator suitable for remote actuation of said pressure exerter, wherein actuation of said actuator remotely causes said pressure exerter to make contact with and exert pressure against the manual ventilator system bag to thereby execute ventilation of a patient associated with the inlet hose.

2. The actuator system of claim 1, wherein the pressure exerter comprises pressure paddles aside the manual ventilator system bag.

3. The actuator system of claim 1, wherein the pressure exerter comprises an air-filled ring around the manual ventilator system bag.

4. The actuator system of claim 1, wherein the actuator comprises a pedal.

5. The actuator system of claim 1, wherein the actuator comprises a handle.

6. The actuator system of claim 1, wherein the actuator comprises a button.

7. The actuator system of claim 1, further comprising a cable within a sheath traversing from said actuator to the manual ventilation system bag.

8. The actuator system of claim 7, further comprising a plurality of springs physically associated with the pressure exerter, wherein actuation of said actuator activates the springs.

9. The actuator system of claim 8, wherein the activation comprises compression of the springs.

10. The actuator system of claim 8, wherein the activation comprises decompression of the springs.

11. The actuator system of claim 7, wherein the cable comprises an air-filled cable comprising at least one valve, wherein actuation of said actuator actuates the at least one valve.

12. An actuator system for actuating a manual ventilator system bag suspended from a bag mount suitable for maintaining a position of the manual ventilator system bag and at least an inlet house associated with the manual ventilator system bag, comprising:
a pressure exerter proximate to the manual ventilator system bag; and
a pedal actuator suitable for remote actuation of said pressure exerter, wherein actuation of said pedal actuator remotely causes said pressure exerter to make contact with and exert pressure against the manual ventilator system bag to thereby execute ventilation of a patient associated with the inlet hose.
